(19) United States
(12) Patent Application Publication
(10) Pub. No.: US 2018/0179512 A1
(43) Pub. Date: Jun. 28, 2018

(54) Novel self-photosensitized nonphotosynthetic microorganism

(71) Applicant: The Regents of the University of California, Oakland, CA (US)

(72) Inventors: Kelsey K. Sakimoto, Cambridge, MA (US); Andrew B. Wong, San Francisco, CA (US); Peidong Yang, Kensington, CA (US)

(73) Assignee: The Regents of the University of California, Oakland, CA (US)

(21) Appl. No.: 15/849,245

(22) Filed: Dec. 20, 2017

Related U.S. Application Data
(60) Provisional application No. 62/438,418, filed on Dec. 22, 2016.

(51) Int. Cl. C12N 11/02; C12N 1/38; C12N 15/03

(52) U.S. Cl. CPC C12N 11/02; C12N 15/03; C12N 1/38

(57) ABSTRACT

The present invention provides for a genetically modified microorganism capable of photosynthesizing an organic compound from carbon dioxide, wherein the microorganism comprises a semiconductor nanoparticle on the surface of the microorganism.

### CROSS-REFERENCE TO RELATED APPLICATIONS

This application claims priority to U.S. Provisional Patent Application Ser. No. 62/438,418, filed on Dec. 22, 2016, which is hereby incorporated by reference.

### STATEMENT OF GOVERNMENTAL SUPPORT

The invention was made with government support under Contract No. DE-AC02-05CH11231 awarded by the U.S. Department of Energy and Grant No. DMR1507914 awarded by the National Science Foundation. The government has certain rights in the invention.

### FIELD OF THE INVENTION

The present invention is in the field of self-photosensitized nonphotosynthetic microorganisms.

### BACKGROUND OF THE INVENTION

The necessity of improving the natural mechanisms of solar energy capture for sustainable chemical production (1) has motivated the development of photoelectrochemical devices based on inorganic solid-state materials (2). Although solid-state semiconductor light absorbers often exceed biological light harvesting in efficiency (3), the transduction of photoexcited electrons into chemical bonds (particularly toward multicarbon compounds from CO₂) remains challenging with abiotic catalysts (4,5). Such catalysts struggle to compete with the high-specificity, low-cost material requirements and the self-replicating, self-repairing properties of biological CO₂ fixation (6). Thus, a viable solution must combine the best of both worlds: the light-harvesting capabilities of semiconductors with the catalytic power of biology.

Several inorganic-biological hybrid systems have been devised: semiconductor nanoparticles with hydrogenases to produce biohydrogen (7), long wavelength absorbing nanomaterials to improve the photosynthetic efficiency of plants (8), and whole cells with photoelectrodes for CO₂ fixation (9, 10). Whole-cell microorganisms are favored to facilitate the multistep process of CO₂ fixation and can self-replicate and self-repair (11). Furthermore, bacteria termed “electrotrophs” can undergo direct electron transfer from an electrode (12). However, traditional chemical synthesis of the semiconductor component often requires high-purity reagents, high temperatures, and complex microfabrication techniques. Additionally, the integration of such foreign materials with biotic systems is nontrivial (13). Many reports have shown that some microorganisms induce the precipitation of nanoparticles (14), producing an inherently biocompatible nanomaterial under mild conditions.

Although photosynthetic organisms can precipitate semiconductor nanoparticles, their metabolic pathways are arguably less desirable than those of their nonphotosynthetic counterparts. Although gene modification of phototrophs has progressed (15), nonphotosynthetic bacteria remain the workhorse of synthetic biology, offering a facile way to tailor the product diversity from CO₂ reduction (16). Additionally, thermodynamic comparisons reveal substantial energetic advantages to photosensitizing nonphotosynthetic CO₂ reduction (17). Of particular interest is the Wood-Ljungdahl pathway, through which CO₂ is reduced to acetyl coenzyme A (acetyl-CoA), a common biosynthetic intermediate, and eventually to acetic acid, both of which can be further upgraded to high-value products by wild-type and genetically engineered organisms (10, 18). This pathway is also used by CO₂-fixing electrotrophs, enabling the use of semiconductor photoelectrons in this energetically efficient biosynthetic route.

### SUMMARY OF THE INVENTION

The present invention provides for a genetically modified microorganism capable of photosynthesizing an organic compound from carbon dioxide, wherein the microorganism comprises a semiconductor nanoparticle on the surface of the microorganism.

The present invention provides for a method for constructing the microorganism of the present invention, comprising: (a) introducing a nucleic acid encoding one or more biosynthetic enzymes into a microorganism wherein the one or more biosynthetic enzymes enable the microorganism to produce the organic compound, (b) introducing the microorganism into a solution comprising a semiconductor source, a carbon source, and a reducing agent, and (c) culturing the microorganism in the solution, such that the microorganism incorporates a semiconductor nanoparticle on the surface of the microorganism.

The present invention provides for a method of producing an organic compound, comprising: (a) providing a genetically modified microorganism of the present invention, (b) exposing the microorganism to sufficient light to cause the semiconductor nanoparticle to generate hydrogen, such that the microorganism uses the generated hydrogen to produce the organic compound. In some embodiments, the light is of a low light intensity at a level about or lower than that described in Example 1.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. M. thermoacetica-CdS reaction schematics. Depiction of the M. thermoacetica-CdS hybrid system, proceeding from the growth of the cells and bioprecipitation (loading) of the CdS nanoparticles (shown in yellow) through photosynthetic conversion of CO₂ (center right) to acetic acid (right).

FIG. 1B. M. thermoacetica-CdS reaction schematics. Pathway diagram for the M. thermoacetica-CdS system. Two possible routes to generate reducing equivalents, [H], exist: generation outside the cell (dashed line) or generation by direct electron transport to the cell (solid line). Hypothesized electron transfer pathways are presented in FIG. 4.

FIG. 2. Electron microscopy of M. thermoacetica-CdS hybrids. (Panel A) SEM image of CdS nanoparticles on M. thermoacetica. (Panel B) High-angle annular dark field (HAADF) STEM image of a single cell, showing clusters across the entire cell surface. (Panel C) HAADF image and EDS mapping showing clusters mainly composed of (Panel D) cadmium and (Panel E) sulfur. Further elemental mapping is provided in FIG. 6. Scale bars in (Panel A) and (Panel B), 500 nm; in (Panel C) and (Panel D), 50 nm.

FIG. 3A. Photosynthesis behavior of M. thermoacetica-CdS hybrids. Photosynthetic production of acetic acid by M. thermoacetica-CdS hybrids and deletional controls. All points and error bars show the mean and error-propagated SD, respectively, of triplicate experiments.

FIG. 3B. Photosynthesis behavior of M. thermoacetica-CdS hybrids. CFU viability assays for M. thermoacetica-CdS hybrids and deletional controls. All points and error bars show the mean and error-propagated SD, respectively, of triplicate experiments.

FIG. 3C. Photosynthesis behavior of M. thermoacetica-CdS hybrids. Rates of acetic acid production and quantum yields for increasing illumination intensities and M. thermoacetica-CdS concentrations. All points and error bars show the mean and error-propagated SD, respectively, of triplicate experiments.

FIG. 3D. Photosynthesis behavior of M. thermoacetica-CdS hybrids. Photosynthetic acetic acid production under low-intensity simulated sunlight with light-dark cycles. All points and error bars show the mean and error-propagated SD, respectively, of triplicate experiments.

FIG. 3E. Photosynthesis behavior of M. thermoacetica-CdS hybrids. Acetic acid production under dark conditions for varying illumination times. The inset shows the relation between illumination time, τ, and acetic acid yield under dark conditions at increasing multiples of τ. All points and error bars show the mean and error-propagated SD, respectively, of triplicate experiments.

FIG. 4. Hypothesized Electron Transfer Pathways for M. thermoacetica/CdS. “A” indicates hydrogen generation at the semiconductor surface, followed by diffusion into the cell. “B” indicates electron transfer directly from semiconductor to surface/membrane bound hydrogenase. “C” indicates electron transfer to membrane bound cytochromes. “D” indicates secretion of a soluble redox mediator. The proposed full and balanced reaction pathway is depicted in FIG. 1B.

FIG. 5. Growth Kinetics for M. thermoacetica/CdS. Growth kinetics obtained by manual cell count for M. thermoacetica grown on 50 mM glucose with and without Cd₂₊ addition at 24 hours.

FIG. 6. Electron Microscopy of M. thermoacetica/CdS Hybrids. (Panel A) Additional SEM images of M. thermoacetica/CdS hybrids showing (Panel B) larger particles obtained after additional ripening. (Panel C) A CdS free cell for reference. (Panel A) Compared with the HAADF micrograph, the cell body can be distinguished by the presence of biologically common elements such as (Panel B) carbon (Panel C) nitrogen (Panel D) phosphorous (Panel E) oxygen (Panel F) chlorine and (Panel G) sulfur. Of note is the formation of nanoparticle clusters with bright Z-contrast on the bacteria composed largely of (Panel H) cadmium and (Panel G) sulfur. (Panels D, G and H) Additionally, a few particles composed of phosphorus, oxygen and cadmium are observed, suggesting the presence of small amounts of Cd₃(PO₄)₂. Scale bars (Panels A to C) 1 μM, (Panels D to K) 100 nm.

FIG. 7. Characterization of CdS Nanoparticles. (Panel A) UV-Vis Spectrum of M. thermoacetica/CdS hybrids demonstrating a band gap, Eg, of 2.51±0.05 eV. (Panel B) XRD reveals peaks assignable to the pattern of wurtzite CdS (reference peaks from JCPDS data card No. 00-041-1049 given as drop lines). These peaks appear broad due to the small size of the nanoparticles.

FIG. 8. ¹H-NMR Spectrum of Photosynthetic Products. (Panel A) Full spectrum shows a prominent solvent peak (H2O). No significant products detected at chemical shifts larger than the solvent peak. (Panel B) Inset of (Panel A) shows the primary product detected is acetic acid (CH₃COOH), with a collection of peaks assignable to Cys/CySS. Trace peaks assignable to ethanol are occasionally observed, but these arise from residual amounts remaining from sterilization techniques.

FIG. 9. CFU and Cell Counts for M. thermoacetica/CdS During Photosynthesis. Data was obtained by the CFU assay and manual counting with a Petroff-Hauser counting chamber as detailed in the Methods section. Measurements were performed in parallel with the data presented in FIGS. 4A and B. Both an increase in CFU mL⁻¹ and cells were observed under illumination. All values are the mean and error propagated standard deviation of triplicate experiments.

FIG. 10. SEM of Photooxidative Damage to M. thermoacetica/CdS. All scale bars 1 μm.

FIG. 11. Culture Tubes of M. thermoacetica/CdS. “A” indicates UDM with 1 mM Cd(NO₃)₂ (no bacteria). “B” indicates UMD with M. thermoacetica 24 hours after inoculation (at point of Cd²⁺ addition). “C” indicates M. thermoacetica/CdS 24 hours after Cd2+ addition (48 hours total culture time). “D” indicates M. thermoacetica/CdS 48 hours after Cd²⁺ addition (72 hours total culture time).

FIG. 12. Spectrum of Blue Light Illumination Source. A Nikon C-HGFI Intensilight mercury lamp with a 435-485 nm filter was used for blue light photosynthesis quantum yield measurements.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

A polynucleotide is “heterologous” to an organism or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

These and other objects, advantages, and features of the invention will become apparent to those persons skilled in the art upon reading the details of the invention as more fully described below.

The present invention provides for a genetically modified microorganism capable of photosynthesizing an organic compound from carbon dioxide, wherein the microorganism comprises a semiconductor nanoparticle on the surface of the microorganism.

In some embodiments, the microorganism is a nonphotosynthetic microorganism in nature. In some embodiments, the microorganism is genetically modified to enzymatically convert a compound which the unmodified microorganism is capable of synthesizing into the organic compound. In some embodiments, the organic compound is a compound that the microorganism does not produce in nature, or is a compound that the unmodified microorganism does not produce. Nucleic acid encoding one or more biosynthetic enzymes, either native or heterologous, to the microorganism, can be stably introduced into the microorganism so that the microorganism can produce one or more organic compounds, such a biofuel, an isoprenoid compound, a fatty acid, and the like. Such enzymes, nucleic acids, and organic compounds are taught in U.S. Pat. Nos. 7,172,886; 7,183,089; 7,192,751; 7,622,282; 7,622,283; 7,667,017; 7,736,882; 7,915,026; 7,985,567; 8,257,957; 8,288,147; 8,420,833; 8,535,916; 8,569,023; 8,828,684, 8,852,902; 9,040,282; 9,109,175; 9,200,298; 9,334,514; 9,376,691; and 9,382,553.

In some embodiments, the microorganism is a prokaryote. In some embodiments, the microorganism is a eubacteria. In some embodiments, the microorganism is a bacterium of the phylum Firmicutes. In some embodiments, the microorganism is a bacterium of the class Clostridia. In some embodiments, the microorganism is a bacterium of the order Thermoanaerobacterales. In some embodiments, the microorganism is a bacterium of the family Thermoanaerobacteriaceae. In some embodiments, the microorganism is a bacterium of the genus Moorrella. In some embodiments, the microorganism is a Moorella thermoacetica or Moorella thermoautotrophica cell.

In some embodiments, the semiconductor nanoparticle is an inorganic semiconductor nanoparticle. In some embodiments, the inorganic semiconductor nanoparticle comprises CdS. In some embodiments, the microorganism can use the semiconductor nanoparticle to fix nitrogen.

In some embodiments, the genetically modified microorganism can carry out one or more of the reactions described in Example 1 and/or the figures.

The present invention provides for a method for constructing the microorganism of the present invention, comprising: (a) introducing a nucleic acid encoding one or more biosynthetic enzymes into a microorganism wherein the one or more biosynthetic enzymes enable the microorganism to produce the organic compound, (b) introducing the microorganism into a solution comprising a semiconductor source, a carbon source, and a reducing agent, and (c) culturing the microorganism in the solution, such that the microorganism incorporates a semiconductor nanoparticle on the surface of the microorganism. In some embodiments, the culturing step further comprises exposing the microorganism to sufficient light to cause the semiconductor nanoparticle to generate hydrogen.

In some embodiments, the method comprises: (a) introducing a nucleic acid encoding one or more biosynthetic enzymes into a microorganism wherein the one or more biosynthetic enzymes enable the microorganism to produce the organic compound, (b) introducing a microorganism into a solution comprising a Cd source, a carbon source, a reducing agent, and a sulfur source, and (c) culturing the microorganism in the solution, such that the microorganism incorporates a CdS nanoparticle on the surface of the microorganism.

In some embodiments, the Cd source is Cd(NO₃)₂. In some embodiments, the carbon source is glucose. In some embodiments, the reducing agent and the sulfur source are both Cys⋅HCl.

In one embodiment, the invention provides for a non-photosynthetic bacterium, such as Moorella thermoacetica, that is induced to precipitate CdS semiconductor nanoparticles. Light adsorption by the CdS enables the bacterium to photosynthetically produce acetic acid from CO₂.

The present invention provides for a method of producing an organic compound, comprising: (a) providing a genetically modified microorganism of the present invention, (b) exposing the microorganism to sufficient light to cause the semiconductor nanoparticle to generate hydrogen, such that the microorganism uses of the generated hydrogen to produce the organic compound. In some embodiments, the light is of a low light intensity at a level about or lower than that described in Example 1.

The genetically modified microorganism can also be used as an optical probe in the study of biological behavior. The genetically modified microorganism can also be used in the synthesis of chemical commodities and feedstocks, carbon sequestration, or waste water treatment.

### REFERENCES CITED

1. A. W. D. Larkum, Curr. Opin. Biotechnol. 21, 271-276 (2010).
2. D. Gust, T. A. Moore, A, L. Moore, Acc. Chem. Res. 42, 1890-1898 (2009).
3. R. E. Blankenship et al., Science 332, 805-809 (2011).
4. T. J. Meyer, Acc. Chem. Res. 22, 163-170 (1989).
5. A. M. Appel et al. Chem. Rev. 113, 6621-6658 (2013).
6. A. S. Hawkins, P. M. McTeman, H. Lian, R. M. Kelly, M. W. W. Adams, Curr. Opin. Biotechnol, 24, 376-684 (2013).
7. K. A. Brown, M. B. Mika, M. Boehm, G. Dukovic, P. W. King, J. Am. Chem. Soc. 134, 5627-5636 (2012).
8. J. P. Giraldo et al. Nat. Mater. 13, 400-408 (2014).
9. J. P. Torella et al., Proc. Natl. Acad. Sci. U.S.A. 112, 2337-2342 (2015)
10. C. Liu et al, Nano Lett. 15, 3634-3639 (2015).
11. L. Lapinsonnière, M. Picot, F. Barribiére, ChemSusChem 5, 995-1005 (2012).
12. K. P. Nevin et al., Appl. Environ. Microbial., 77, 2882-2886 (2011).
13. P. W. King, Biochem. Biophys. Acta 1827, 949-957 (2013).
14. W. J. Crookes-Goodson, J. M. Slocik. R. R. Naik, Chem. Soc. Rev. 37, 240-2412 (2008).
15. D. C. Ducat, P. A. Silver, Curr. Opin. Chem. Biol. 16, 337-344(2012).
16. L. S. Gronenberg, R. J. Marcheschi, J. C. Liao, Curr. Opin. Chem. Biol. 17, 462-471 (2013).
17. A. G. Fast, E. T. Papoutakis, Curr. Opin. Chem. Eng. 1, 380-395 (2012).
18, M. C. A. A. Van Eerten-Jansen et al., ACS Sustainable Chem. Eng. 1, 513-518 (2013).
19. Materials and methods are found in Example 1 herein.
20. R. Vogel, P. Hoyer, H. Weller, J. Phys. Chem. 98, 3183-3188 (1994).
21. H. L. Drake, S. L. Daniel, Res. Microbiol. 155, 869-883 (2004).
22. D. P. Cunningham, L. L. Lundie Jr., Appl. Environ. Microbiol. 59, 7-14 (1993).
23. J. D. Holmes et al., Photochem. Photobiol. 62, 1022-1026 (1995).
24. S. L Daniel, T. Hsu, S. I. Dean, H. L. Drake, J. Bacteriol, 172, 4464-4471 (1990).
25. E. Dumas et al., Environ. Sci. Technol. 44, 1464-1470 (2010).
26. B. Liu, X. Zhao, C. Terashima, A. Fujishima, K. Nakata, Phys. Chem. Chem. Phys. 16, 8751-8760 (2014).
27. K. Schuchmann, V. Müller, Nat. Rev. Microbiol. 12, 809-821 (2014).
28. B. Hankamer et al., Physiol. Plant. 131, 10-21 (2007).
29. H. G. Cha, K.-S, Choi, Nat. Chem. 7, 328-333 (2015).
30. B. E. Logan, K. Rabaey, Science 337, 686-690 (2012).
31. A. Kita et al., J. Biosci. Bioeng. 115, 347-352 (2013).
32. H. M. Jensen et al., Proc. Natl. Acad. Sci. U.S.A. 107, 19213-19218 (2010).
33. C. L. Wang, A. M. Lurn, S. C, Ozuna, D. S. Clark, J. D. Keasling, Appl. Microbiol. Biotechnol. 56, 425-430 (2001).
34. M. Rosenbaum, F. Aulenta, M. Villano, L. T. Angenent, Bioresour. Technol. 102, 324-133 (2011).
35. J. S. Deutzmann, M. Sahin, A. M. Spormann, mBio. 6, e00496-15 (2015).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### EXAMPLE 1

### Self-Photosensitization of Nonphotosynthetic Bacteria for Solar-to-Chemical Production

Improving natural photosynthesis can enable the sustainable production of chemicals. However, neither purely artificial nor purely biological approaches seem poised to realize the potential of solar-to-chemical synthesis. We developed a hybrid approach, whereby we combined the highly efficient light harvesting of inorganic semiconductors with the high specificity, low cost, and self-replication and -repair of biocatalysts. We induced the self-photosensitization of a nonphotosynthetic bacterium, Moorella thermoacetica, with cadmium sulfide nanoparticles, enabling the photosynthesis of acetic acid from carbon dioxide. Biologically precipitated cadmium sulfide nanoparticles served as the light harvester to sustain cellular metabolism. This self-augmented biological system selectively produced acetic acid continuously over several days of light-dark cycles at relatively high quantum yields, demonstrating a self-replicating route toward solar-to-chemical carbon dioxide reduction.

We developed a hybrid system containing the nonphotosynthetic CO₂-reducing bacterium Moorella thermoacetica (ATCC 39073) and its biologically precipitated CdS nanoparticles (19). CdS is a well-studied semiconductor with an appropriate band structure and is suitable for photosynthesis (20). As an acetogen and an electrotroph, M. thermoacetica serves as an ideal model organism to explore the capabilities of a hybrid system (21).

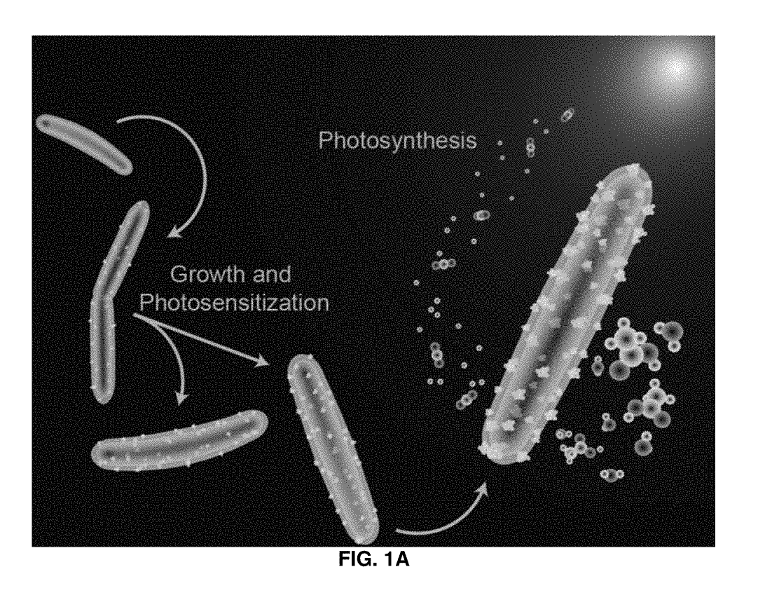

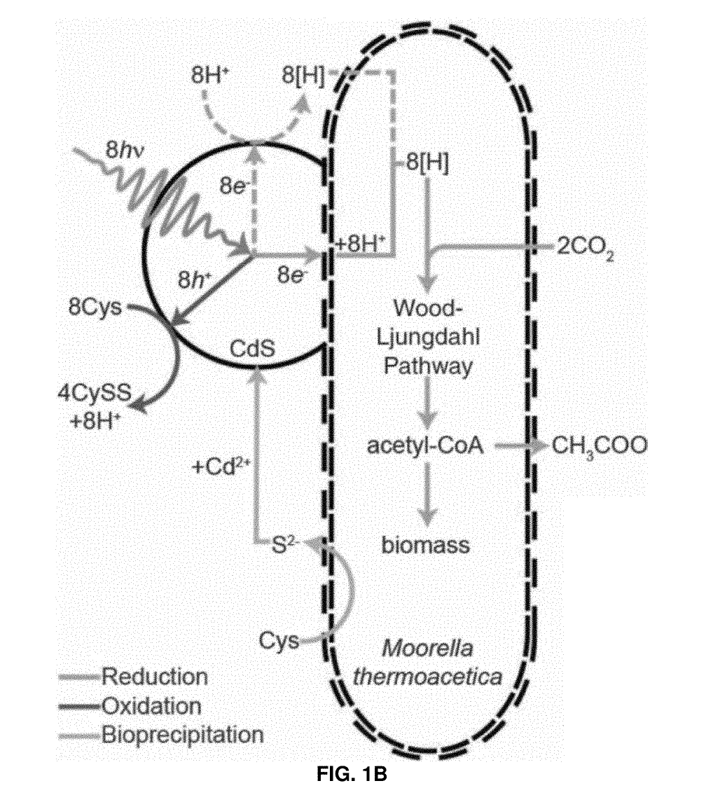

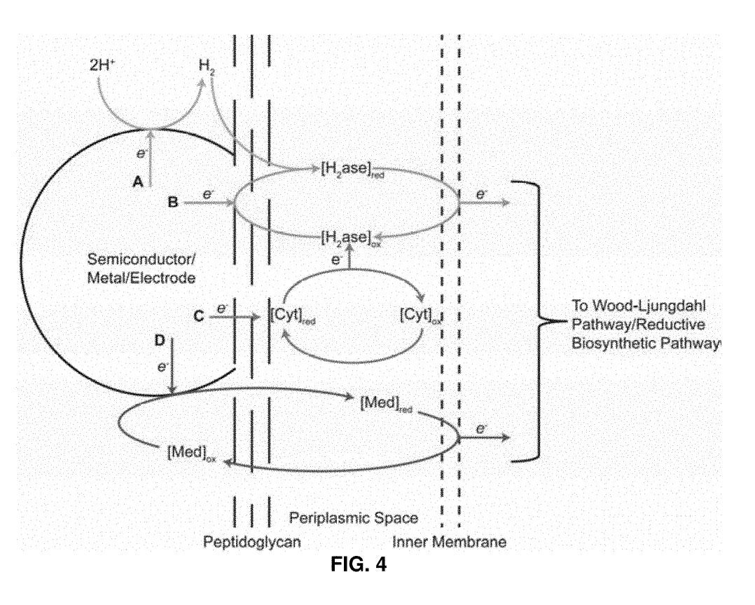

The photosynthesis of acetic acid by M. thermoacetica and CdS is a two-step, one-pot synthesis (FIGS. 1A and 1B). First, the precipitation of CdS by M. thermoacetica is triggered by the addition of Cd²⁺ and cysteine (Cys) as the sulfur source (19, 22). M. thermoacetica uses photogenerated electrons from illuminated CdS nanoparticles to carry out photosynthesis (FIG. 1B). The absorption of a photon, hv, by CdS produces an electron and hole pair, e⁻ and h⁺. The electron generates a reducing equivalent, [H] (see FIG. 4), that is passed on to the Wood-Ljungdahl pathway to synthesize acetic acid from CO₂. Cysteine quenches the h⁺, leading to the oxidized disulfide form, cystine (CySS). The overall photosynthetic reaction is

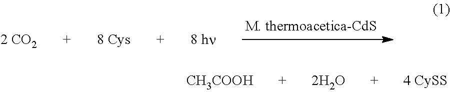

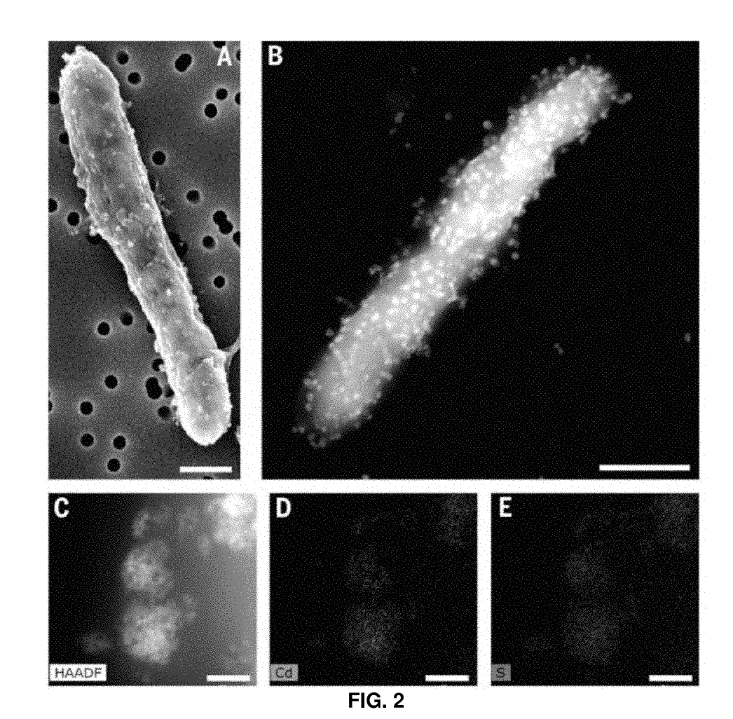

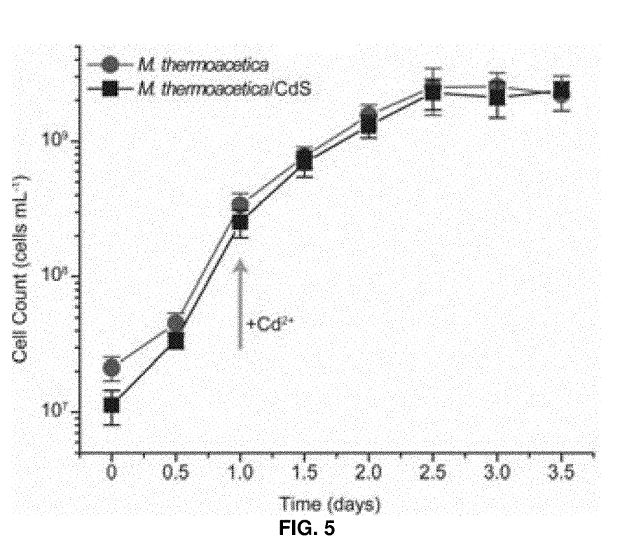

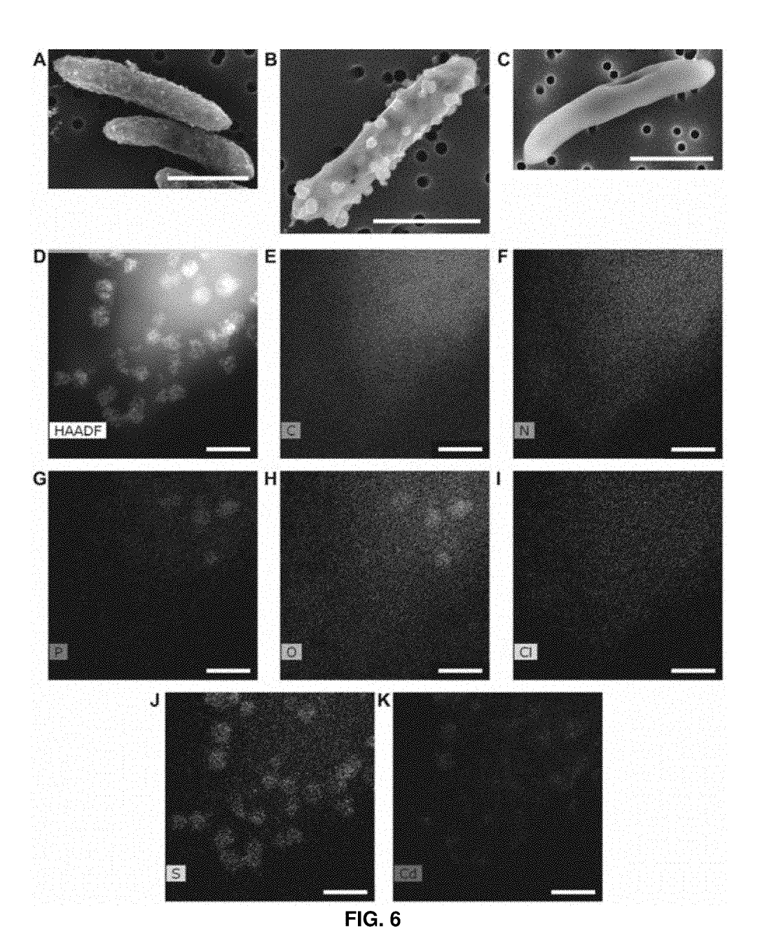

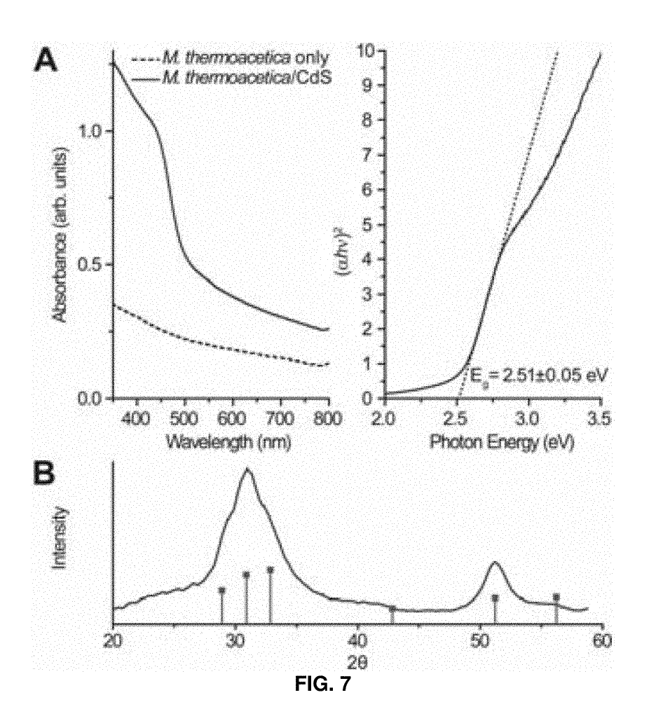

The precipitation of CdS by M. thermoacetica was initiated by the addition of Cd(NO₃)₂ to an early exponential growth culture of glucose-grown cells supplemented with Cys (FIG. 5) (19). Scanning electron microscopy (SEM), scanning transmission electron microscopy (STEM), and energy-dispersive x-ray spectroscopy (EDS) mapping revealed clusters of smaller nanoparticles (<10 nm; FIGS. 2A and 2B) composed of cadmium and sulfur on the cells (FIG. 2, Panels C to E, and FIG. 6). Absorption spectra and Tauc plots yielded a measured direct band gap of 2.51±0.05 eV (FIG. 7, Panel A). The slightly larger measured band gap relative to bulk CdS (2.42 eV) suggests the quantum confinement expected of <10-nm particles (20). Powder x-ray diffraction showed broad peaks consistent with small particles of the wurtzite phase (FIG. 7, Panel B).

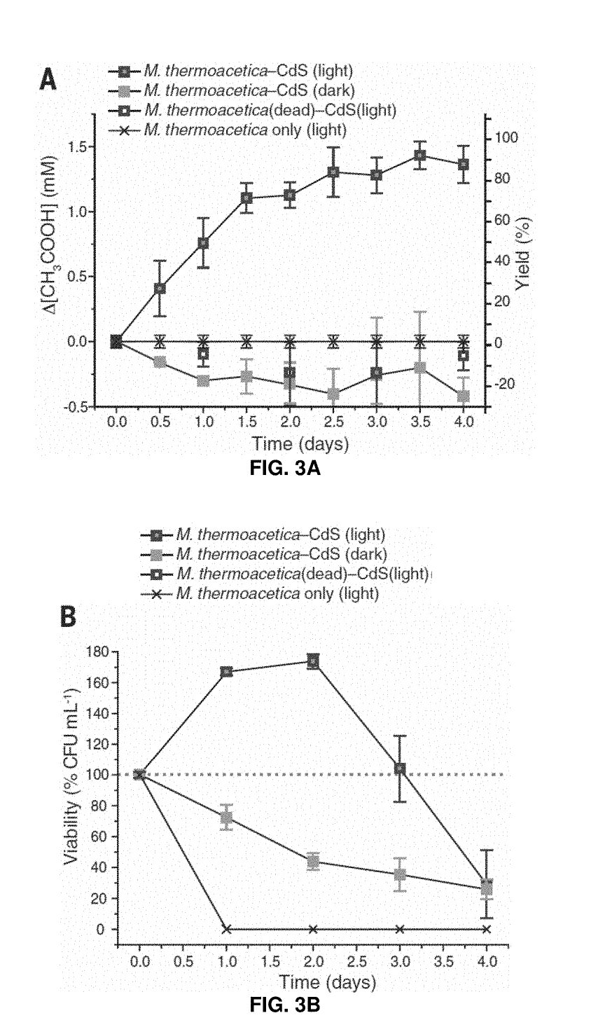

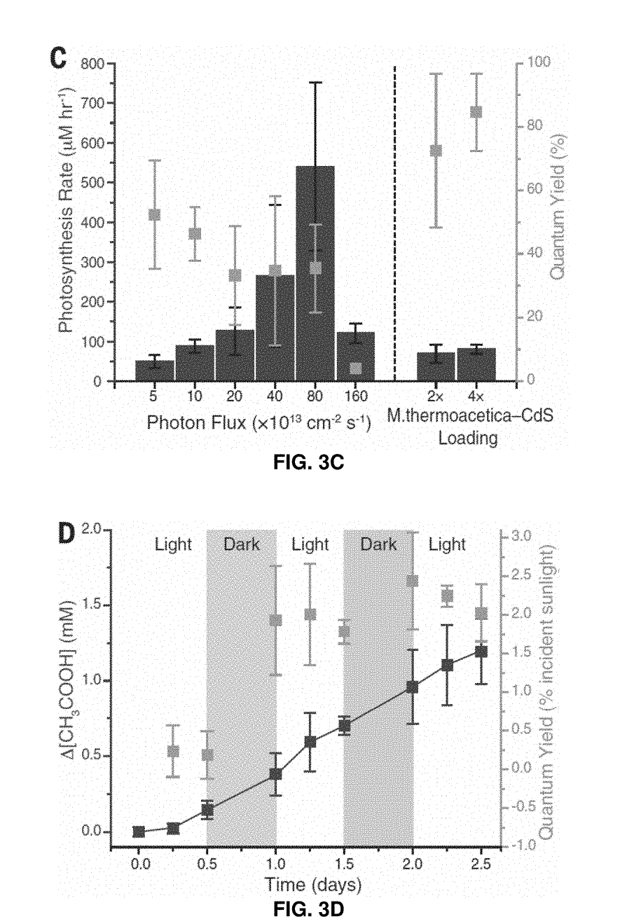

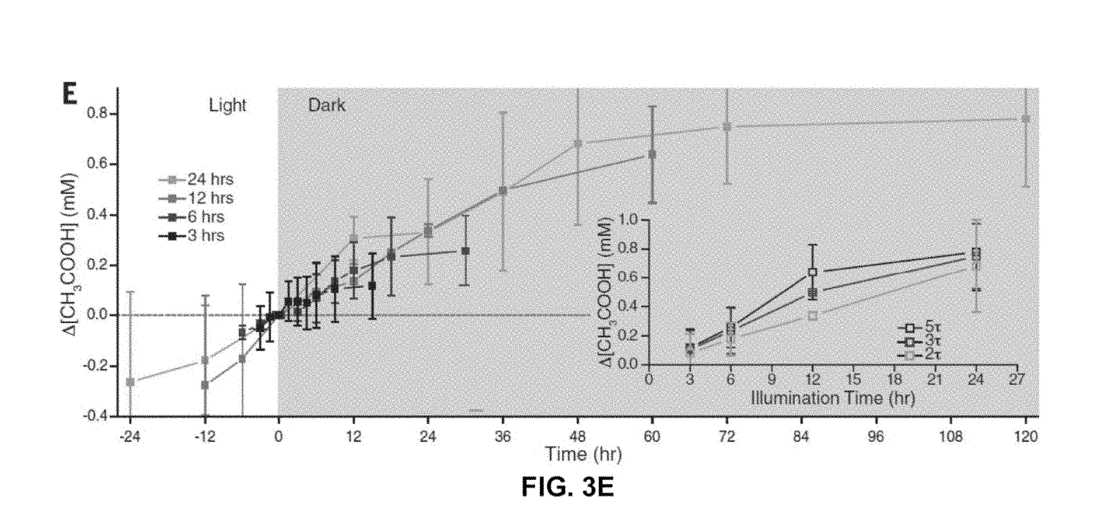

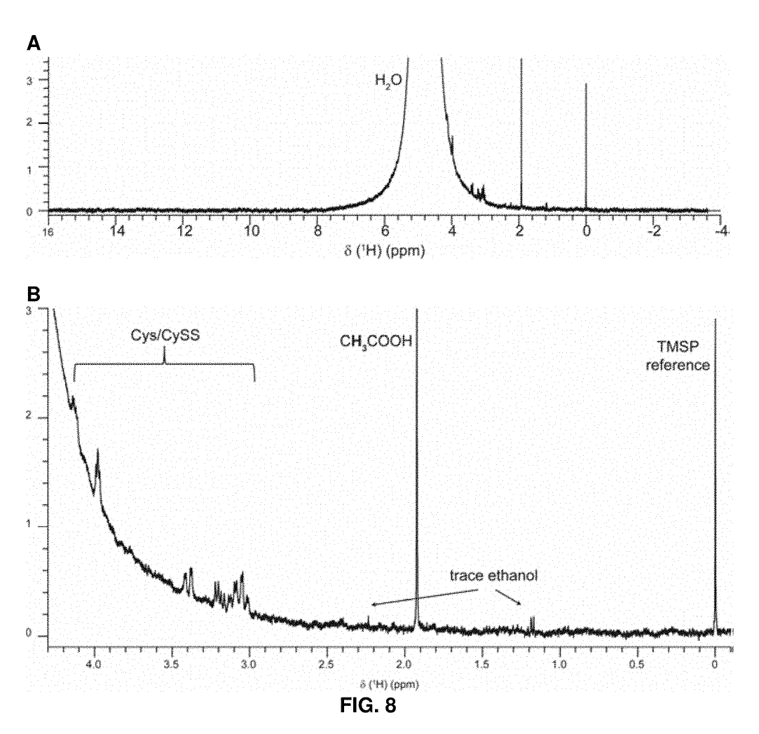

To confirm photosynthesis, a series of deletional control experiments was carried out in which M. thermoacetica, CdS, and light were systematically removed (FIGS. 3A and 3B). In the absence of light (405±5 nm), acetic acid concentrations decreased [from 2 mM accumulated under initial H₂:CO₂ acclimation, as measured by quantitative proton nuclear magnetic resonance (¹H-qNMR) spectroscopy], potentially as the result of a dark catabolic process. The viability determined by colony-forming units (CFU) assays slowly declined to ˜25% after 4 days (from the initial 5.9±0.4×10⁴ CFU ml⁻¹ and 1.7±0.4×10⁹ cells ml⁻¹), indicating that the M. thermoacetica-CdS system requires light to maintain viability. The viability of bare M. thermoacetica without CdS dropped to 0% within the first day under light, consistent with previous observations of semiconducting and/or insulating precipitates having a photoprotective role toward bacteria (23). Only M. thermoacetica-CdS hybrids exposed to light produced acetic acid. The ¹H-qNMR spectrum revealed acetic acid to be the only product of CO₂ reduction, confirming the high selectivity expected of biological catalysts (FIG. 8). After the first 1.5 days, the rate of production began to plateau because of the limiting amounts of the sacrificial reductant, Cys.

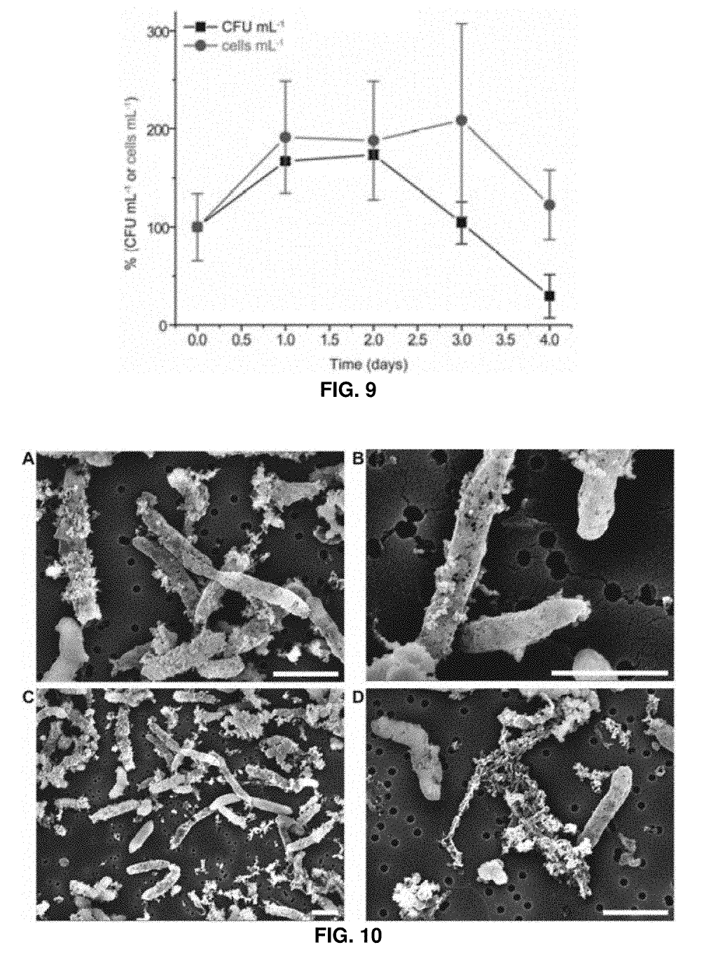

We calculated a maximum yield of 90% acetic acid (based on the initial Cys concentration), which is consistent with previous observations that in the Wood-Ljungdahl pathway, ˜10% of reduced CO₂ is directed toward cell biomass (24). During photosynthesis, both the viability and the cell counts of the M. thermoacetica-CdS system nearly doubled after the first day (FIG. 9), on par with the doubling time of autotrophic growth (˜25 hours). Although the growth was not vigorous and perhaps was limited by the total amount of CdS and other nutrients, these results suggest the possibility of a completely self-reproducing hybrid organism sustained purely through solar energy. After the third and fourth days, viability decreased in coincidence with the depletion of Cys, leading to oxidative photodamage (FIG. 10).

Under increasing blue light flux (435 to 485 nm), the rate of acetic acid production increased (FIG. 3C). At 5×10¹³ photons cm⁻² s⁻¹, a quantum yield of 52±17% was observed. The rate of photosynthesis increased up to 160×10¹³ photons cm⁻² s⁻¹, after which the rate dramatically decreased and the quantum yield dropped to 4±1%, possibly due to photooxidative degradation under high light intensities (25). At high light fluxes, large holes formed in the cell surface and, in some cases, resulted in the complete destruction of the cell membrane (FIG. 10). The high quantum yield of the M. thermoacetica-CdS system is notable, given that previous analogous systems often have had reported quantum efficiencies of ˜20% (7). This result is rationalized by the low light flux of these measurements, which reduces losses from recombination (26). With four times the normal loading of M. thermoacetica-CdS hybrids, we measured a quantum yield of 85±12% (FIG. 3C). Under higher concentrations, the average flux per bacterium decreased, correlating with increased quantum yield.

To further characterize their photosynthetic behavior, we illuminated M. thermoacetica-CdS hybrids under low-intensity simulated sunlight (air mass 1.5 global spectrum, 2 W m⁻²) with a light-dark cycle of 12 hours each to mimic day-night cycles (FIG. 3D). Unexpectedly, acetic acid concentrations not only increased under illumination but continued to increase in the dark at the same rate, through several light-dark cycles. A potential explanation lies in the accumulation of biosynthetic intermediates during the light cycle, which are then used during the dark cycle. These may include a number of reductive species [e.g., NADH (reduced nicotinamide adenine dinucleotide), NADPH (reduced NAD phosphate), or ferredoxin)] or intermediates in the Wood-Ljungdahl pathway such as acetyl-CoA (27). A proton gradient may also be storing energy for adenosine triphosphate synthesis in the dark. Further experiments that varied the duration of the light cycle (FIG. 3E) revealed a proportionality between the length of illumination, τ, and the acetic acid yield under dark conditions. Although the initial rate during and just after illumination appears to be relatively constant, consistent with a zero-order catalytic reaction, the yield begins to plateau after 2τ, exhibiting a linearity between illumination time and acetic acid yield (FIG. 3E). However, at 5τ, samples that were illuminated for 24 hours break this trend. These observations suggest that during up to 12 hours of illumination, some intermediate accumulates, enabling a proportional acetic acid yield during the dark cycle. Beyond this, the intermediate may saturate, with longer illumination times yielding no further acetic acid. We measured a peak quantum yield of 2.44±0.62% of total incident low-intensity simulated sunlight (FIG. 3D). These quantum yields are order-of-magnitude comparable to the year-long averages determined for plants and algae, which range from ˜0.2 to 1.6% (1).

Biological routes to solid-state materials have often struggled to compete with high-quality traditionally synthesized materials. This work demonstrates not only that biomaterials can be of sufficient quality to carry out useful photochemistry, but that in some ways they may be more advantageous in biological applications. Most traditional nanoparticle syntheses require organic capping ligands to control the particle shape. These ligands present a barrier to charge transfer between the semiconductor and the catalyst, often requiring electron tunneling (13). The ligand-free approach taken here may help to establish a favorable interface between the bacteria and the semiconductor, resulting in improved efficiencies. Additionally, metal chalcogenides such as CdS have had limited application because of oxidative photodegradation; the ability of bacteria to precipitate metal chalcogenides from the products of photodis solution (Cd²⁺ and oxidized sulfur complex ions) suggests a potential regenerative pathway to circumvent the debilitating photoinstability through a precipitative self-regeneration.

The M. thermoacetica-CdS system displays behavior that may help it to exceed the utility of natural photosynthesis. First, the quantum yield increased with higher M. thermoacetica-CdS concentrations. The ability to tune the effective light flux per bacterium by changing the concentration of the suspension is a considerable advantage over similar light management practices in natural photosynthesis that are achieved through genetic engineering of chloroplast expression (28). Second, the catabolic energy loss observed during dark cycles in natural photosynthesis was absent in our hybrid system, which may be an innate feature of the Wood-Ljungdahl pathway, in which acetic acid is a waste product of normal respiration. Additionally, many plants and algae tend to store a large portion of their photosynthetic products as biomass, which requires extensive processing to produce useful chemicals. In contrast, the M. thermoacetic-aCdS system directs ˜90% of photosynthetic products toward acetic acid, reducing the cost of diversifying to other chemical products.

This system could be improved by substituting Cys oxidation with a more beneficial oxidation reaction, such as oxygen evolution, wastewater oxidation for water purification, or oxidative biomass conversion (29, 30). Expanding the material library available through biologically induced precipitation will increase the capacity for light absorption and raise the upper limit on semiconductor-bacteria photosynthetic efficiency. The availability of genetic engineering tools for M. thermoacetica (31), as well as the introduction of electrotrophic and nanoparticle precipitation behavior in model bacteria such as Escherichia coli (32, 33), suggests a potential role for synthetic biology in rationally designing such hybrid organisms.

Beyond the development of advanced solar-to-chemical synthesis platforms, this hybrid organism also has potential as a tool to study biological systems. The native integration of semiconductor nanoparticles with bacterial metabolic processes provides a distinctive optical tag for the study of microbial behavior, such as semiconductor-bacteria electron transfer (34, 35), by providing a sensitive, noninvasive, nonchemical probe.

### Materials and Methods

Growth of Moorella thermoacetica/Cadmium Sulfide:

Anaerobic media was prepared with three formulations in deionized (DI) water: undefined precipitation medium (UPM), defined photosynthesis medium (DPM) and undefined solid media (USM) as given below:

| | UPM | DPM | USM |
|---|---|---|---|
| Component | (g L⁻¹) | (g L⁻¹) | (g L⁻¹) |
| Salt Mix: | | | |
| NaCl | 0.40 | 0.40 | 0.40 |
| NH₄Cl | 0.40 | 0.40 | 0.40 |
| MgSO₄•7H₂O | 0.33 | 0.33 | 0.33 |
| CaCl | 0.05 | 0.05 | 0.05 |
| KCl | 0.25 | 0.25 | 0.25 |
| Phosphorus Source: | | | |
| K₂HPO₄ | — | 0.64 | 0.64 |
| β-glycerophosphate•2Na•xH₂O | 0.80 | — | — |
| Buffer: | | | |
| NaHCO₃ | 2.50 | 2.50 | 2.50 |
| Supplemental Components: | | | |
| Wolfe's Vitamin Mix* | 10 mL | 10 mL | 10 mL |
| Trace Mineral Mix** | 10 mL | 10 mL | 10 mL |
| Yeast Extract | 0.50 | — | 2.00 |
| Tryptone | 0.50 | — | 2.00 |
| Agar | — | — | 20.00 |

*From filter sterilized stock solution containing (mg L⁻¹ DI):

| | |
|---|---|
| Pyridoxine•HCl | 10.0 |
| Thiamine•HCl | 5.0 |
| Riboflavin | 5.0 |
| Nicotinic acid | 5.0 |
| Calcium D-(+)-pantothenate | 5.0 |
| Thioctic acid | 5.0 |
| Biotin | 2.0 |
| Folic acid | 2.0 |
| Vitamin B12 | 0.1 |

**From filter sterilized stock solution containing (mg L⁻¹ DI):

| | |
|---|---|
| Nitriloacetic acid | 2000.0 |
| MnSO₄•H₂O | 1000.0 |
| Fe(SO₄)₂(NH₄)₂•6H₂O | 800.0 |
| CoCl₂•6H₂O | 200.0 |
| ZnSO₄•7H₂O | 0.2 |
| CuCl₂•2H₂O | 20.0 |
| NiCl₂•6H₂O | 20.0 |
| Na₂MoO₄•2H₂O | 20.0 |
| Na₂SeO₄ | 20.0 |
| Na₂WO₄ | 20.0 |

Yeast extract, tryptone, and agar were obtained from BD Biosciences, all other reagents were obtained through Sigma-Aldrich.

All gases were passed through two in-line oxygen traps (Agilent Technologies) to render anoxic prior to use.

Media (UPM, DPM, USM) were prepared by boiling an appropriate volume (100-500 mL) of DI water, salt mix, and trace mineral mix for 5 min under a continuously purged N₂ atmosphere. An additional 10 vol. % of DI was added to account for evaporation losses during boiling. The solution was then cooled to room temperature in an ice bath, and the atmosphere was switched to an 80:20 mixture of N₂:CO₂. The phosphorus source, buffer, and supplemental components were then added, and the media was allowed to purge and equilibrate at room temperature for 20 min. No adjustment of pH was necessary. Anaerobic media was then dispensed under the same atmosphere into 18×150 mm Balch-type anaerobic culture tubes (Chemglass Life Sciences) with butyl stoppers and aluminum crimp seals. Media was then autoclaved for 15 min at 121° C.

Stock solutions of 1 M glucose as a carbon source, 5.0 wt. % Cys-HCl as a reducing agent and sulfur source, and 100 mM Cd(NO₃)₂ as a cadmium source were prepared by boiling DI water for 5 min under N₂, cooling to room temperature, dissolving the respective solutes, and sealing under N₂ in 200 mL serum bottles with butyl stoppers and aluminum crimp seals. Stock solutions of glucose and Cys were autoclaved for 15 min at 121° C. prior to use. All concentrations of glucose, Cys, and cadmium are given nominally, calculated from the initial volume of the media.

The Hungate technique or an anaerobic chamber (Coy Laboratory Products, Inc.) was employed in all operations to prevent exposure of the anaerobic bacteria to oxygen.

The initial inoculum of Moorella thermoacetica ATCC 39073 was graciously obtained from the laboratory of Prof. Michelle C. Y. Chang and recultured in UPM supplemented with 50 mM glucose and 0.1 wt. % cysteine. Late log cultures were cryopreserved in liquid nitrogen with 10% dimethylsulfoxide as a cryoprotectant.

To prepare M. thermoacetica/CdS hybrids, 20 mL UPM supplemented with 25 mM glucose and 0.1 wt. % cysteine was inoculated from the thawed cryopreserved stock at 5 vol. % and incubated with occasional agitation at 52° C. The headspace of each tube was pressurized to 150 kPag with 80:20 N₂:CO₂.

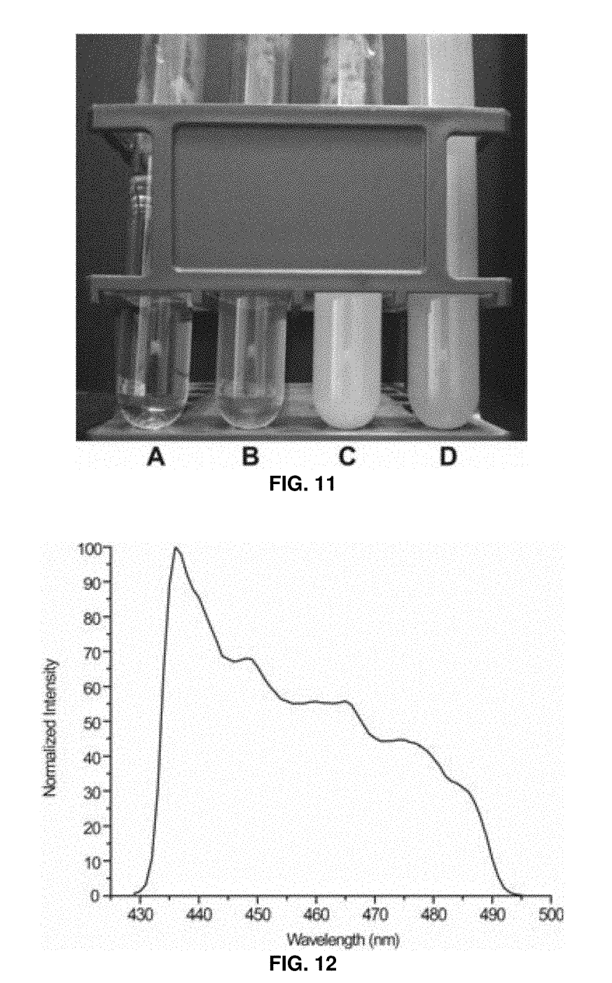

After 3 days of growth (OD₆₀₀=0.16), the culture was reinoculated at 5 vol. % into fresh UPM (50 mM glucose, 0.1 wt. % cysteine), and incubated at 52° C. After 24 hours, 1 mM Cd(NO₃)₂ was added to the tubes. Each tube was returned to incubation and placed in an in-house fabricated rotator that slowly inverted each tube at a continuous rate of 4.5 min⁻¹. The suspension turned initially opaque white due to the formation of quantum confined CdS nanoparticles, and eventually ripened into an opaque yellow suspension (FIG. 11).

After an additional 2 days (3 days total growth), each tube was centrifuged at 2500 rpm for 30 min, washed and resuspended in an equivalent volume of DPM supplemented with 0.1 wt. % cysteine. A volume of 10 mL of the suspension was distributed into clean anaerobic tubes equipped with a magnetic stir bar (sealed and autoclaved as previously described). Each tube was pressurized with 150 kPag of 80:20 H₂:CO₂ and incubated for 12-24 hours at 52° C. to promote autotrophic respiration.

Material Characterization of M. thermoacetica/CdS: The UV-Vis spectra of M. thermoacetica/CdS suspensions were obtained with a Shimadzu UV3101PC UV-Vis-NIR Spectrophotometer with an integrating sphere. Suspensions were diluted 8× in DI for measurements to ensure linearity under the Beer-Lambert law assumption. Band gap measurements were generated from Tauc plots assuming a 1 cm path length for each cuvette and a direct band gap transition for CdS. Samples for XRD were prepared by drop casting M. thermoacetica/CdS suspensions onto quartz chips and air drying. XRD patterns were measured with a Bruker AXS D8 Advance Diffractometer with a Co Kα source.

Electron Microscopy: Samples of M. thermoacetica/CdS were prepared for electron microscopy by fixing overnight in a solution of 5 vol. % glutaraldehyde in DPM at a 50:50 ratio of DPM/glutaraldehyde:M. thermoacetica/CdS suspension. Fixed samples were prepared for SEM by vacuum filtering onto 0.1 μm Isopore polycarbonate membrane filters (EMD Millipore) previously sputtered with 6 nm of gold. Samples were washed by increasing concentrations of ethanol in DI water (0%, 25%, 50%, 75%, 90%, 100%, ˜10 mL or 10 min each). Membrane filters were finally transferred into hexamethyldisilazane for 1 hour, then air dried overnight. An additional ˜3 nm of gold was sputtered onto the samples prior to SEM mounting and imaging at 5 kV by field emission SEM (JEOL FSM6430). Samples of fixed M. thermoacetica/CdS were prepared for STEM by dropping the fixed suspension onto Formvar coated Ultrathin Carbon Cu TEM grids, settling for 1 hour, and washing briefly in DI water. Grids were air dried overnight. STEM imaging and EDS mapping was performed at 80 kV with an FEI Titan microscope at the National Center for Electron Microscopy (NCEM). The EDS signal was acquired via a FEI Super-X Quad windowless detector based on silicon drift technology that was controlled by Bruker Esprit software.

Photosynthesis Measurements: All photosynthesis measurements were conducted with the suspensions prepared as previously specified. Prior to photosynthesis, an additional 0.1 wt. % cysteine was added to each tube (total 0.2 wt. %, nominal) in part to act as a sacrificial reducing agent. Each tube was stirred magnetically at 150 rpm and heated to a measured temperature of 55° C. by means of a stirring hot plate and shallow sand bath (<1 cm). Three illumination sources were employed: for high-throughput measurements: 1) an in-house fabricated circular LED array composed of 405±5 nm violet LEDs with a measured photon flux of 5×10¹⁸ cm⁻²s⁻¹, for blue light measurements, 2) a collimated Nikon C-HGFI Intensilight mercury lamp with a 435-485 nm filter (FIG. 12), and for simulated sunlight measurements, 3) a collimated 75 W Xenon lamp (Newport, Corp.) with an AM 1.5G filter. All light intensities were calibrated by a silicon photodiode (Hamamatsu S1787-04). Concentrations of photosynthesis products were measured by ¹H-qNMR with sodium 3-(trimethylsilyl)-2,2′,3,3′-tetradeuteropropionate (TMSP-d4, Cambridge Isotope Laboratories, Inc.) as the internal standard in D₂O. Spectra were processed using Mnova NMR. Headspace gas was periodically monitored by gas chromatography with an Agilent 3000A Micro GC (Ar carrier gas, molecular sieve column, thermal conductivity detector).

Colony Forming Unit Assay: Colony Forming Unit Assays were performed by sampling and inoculating 0.1 mL of the M. thermoacetica/CdS photosynthesis suspension into 5 mL of molten (T>50° C.) USM supplemented with 40 mM glucose and 0.1 wt. % cysteine. After thorough mixing, molten agar anaerobic tubes were quickly spun horizontally in ice water to set a thin layer of agar on the sidewalls of the anaerobic tubes. Assay tubes were pressurized to 150 kPag with 80:20 N₂:CO₂ and incubated vertically at 52° C. After 3.5 days of growth, visible white, circular colonies were counted to determine the CFU mL⁻¹ as a measure of cell number and viability. In parallel, cell mL₋₁ values were determined by manual counting with a Petroff-Hauser counting chamber.

Calculations: Photosynthetic acetic acid yields and efficiencies were calculated based on Eq. 6. The 0.2 wt. % Cys-HCl (MW=157.62 g mol₋₁) used in photosynthesis measurements, corresponds to 12.2 mM Cys. With the above stoichiometry, this leads to a maximum acetic acid yield of 1.53 mM CH₃COOH. Acetic acid yield is then defined as:

$${Yield}_{a} ( \% ) = \frac{{C}_{a} ( mM )}{1.53 mM} \times 100 \%$$

where C_{α} is the concentration of acetic acid.

The quantum yield was determined by comparison of the initial rate of acetic acid production with the measured photon flux. The reduction of two CO₂ molecules to one acetic acid molecule requires 8 electrons, giving the following quantum yield (QY) equation:

$$QY ( \% ) = \frac{8 \times {C}_{a} / t \times A \times V \times {N}_{A}}{{Φ}_{ph}}$$

where t is the reaction time to produce C_{α}, A is the area of illumination, V is the volume of the M. thermoacetica/CdS suspension, Φₚₕ is the measured photon flux (photons cm⁻² s⁻¹), N_{A} is Avogadro's Number (6.022×10²³). For a lower limit of the efficiency, and to account for the possibility of scattering and reflections due to the curved sidewalls of the tubular reactor, A was assumed to be the entire surface of the reactor, or 25.4 cm² for a suspension volume of 10 mL.

All reported error bars represent one standard deviation from the mean average obtained from each experiment run in triplicate. All error bars on calculated values obtained after averaging were determined by the standard propagation of errors methodology.

M. thermoacetica/CdS Reaction Equations:

CdS precipitation by M. thermoacetica:

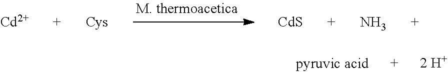

Electron-hole pair photogeneration:

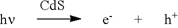

Reducing equivalent generation:

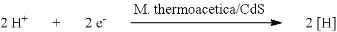

CO₂ reduction to acetic acid:

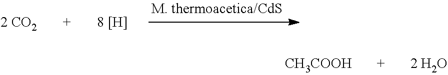

Cys oxidation to CySS:

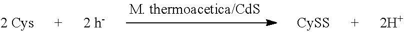

Overall photosynthetic reaction:

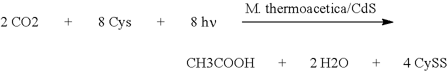

Discussion of M. thermoacetica/CdSgrowth and bioprecipitation: Attempts to establish M. thermoacetica/CdS under different growth substrates yielded mixed results. In the same media, M. thermoacetica failed to produce CdS precipitates when grown purely on a mixture of H₂:CO₂ (80:20), the normal autotrophic growth substrate. When grown on a combination of H₂:CO₂ and glucose, formation of opaque yellow CdS occurred at a slower rate. These observations suggest that the precipitation of CdS depends heavily on the metabolic rate, pathway (fermentative glycolysis versus autotrophic respiration), protein expression under different growth substrates, or a combination of several factors. The influences of the growth substrate and the biological/chemical factors governing the formation of CdS bear further investigation to shed light on the nature of bioprecipitation processes.

Experiments in which dilute suspensions (˜10-50% regular cell density) were supplemented with Cd²⁺ ions and subjected to illumination, a white precipitate was observed (which did not fully ripen to the opaque yellow suspension), suggesting photosynthetic precipitation of CdS. This contrasts sharply with the previous observation that Cd²⁺ precipitation did not occur during autotrophic growth (H₂:CO₂), the presumed pathway for photosynthetic acetic acid synthesis. The difference in biologically-induced precipitation behavior between autotrophic and photosynthetic metabolism bears further investigation.

Discussion of CFU Assays, Viability, and Self-replication: As noted herein, CFU assays suggested that the photosynthetic production of acetic acid was sufficient to produce cell-replication, as evidenced by the increase in CFU mL⁻¹. It is important to note that CFU assays are not definitive, and the observed increase could be an artifact of culturing and growth conditions. To address these issues, we have cross correlated this observation with manual cell counting (Petroff-Hauser counting chamber). The rough doubling measured by CFU mL⁻¹ (167±2% after the first day) correlates reasonably well with the parallel cell count (191±57% after the first day). While the CFU mL⁻¹ begins to plateau after the second day, so does the cells mL⁻¹ (FIG. 10). While the CFU mL⁻¹ begins to decline on the third day, the cells mL⁻¹ does not decline until the fourth day. This is likely due to the initial cell death, followed gradually later by the photooxidative degradation (FIG. 10), which would result in an apparent decrease in the CFU mL⁻¹ and then cells mL⁻¹, respectively.

This cross-correlation serves to rule out a few possibilities and artifacts. Firstly, no sporulation was observed at any time, suggesting that the formation of a light or nutrient deficit triggered dormant state did not occur. While this may still imply the formation of resting cells normal in appearance, which may result in an apparent increase in CFU mL⁻¹, this would also appear as a constant cells mL⁻¹, which was not observed. Conversely, an apparent rise in cells mL⁻¹ could be attributable to simply the counting of dead cells (cell staining with propidium iodide was unable to differentiate between healthy cells and ethanol killed cells). However, this would give a constant CFU mL⁻¹, which was also not observed.

It is possible that the observed cell doubling is simply due to the facile division of cells poised at the end of their division cycle. However, in order to observe the doubling seen here, that would require that nearly every cell had already doubled in cell length (but not yet divided) before illumination. We observed no such features, with a vast majority of cells existing as single cells over all time points. This suggests that cell wall biosynthesis had to predominantly occur under illumination, where light was required for acetic acid metabolism (FIGS. 3A and 3B).

While the growth is not robust (as would be suggested by several doubling cycles), our observations suggest a direct correlation between M. thermoacetica/CdS illumination and their apparent increase in both cell number and cell viability. In experiments inoculated with 50% the normal M. thermoacetica/CdS loading (2× diluted), cell numbers tripled over the first 24 hours (292±69%). It is possible that the initially high concentration of bacteria quickly exhausts a limiting nutrient in the defined photosynthesis medium, allowing only one cell doubling. As noted previously, photosynthesis was not sufficient to produce fully ripened (yellow) CdS nanoparticles. It is possible the upper limit on doubling cycles is set by the CdS loading amount, in which each doubling cycle halves the average nanoparticle cell⁻¹ loading. These initial insights demonstrate that a fully self-replicating hybrid organism may be possible, but will require significant optimization to fully understand and rigorously demonstrate the process.

Headspace Gas Analysis During Photosynthesis: One of the possible electron transfer mechanisms presented in FIG. 4 denotes that the formation of molecular hydrogen, H₂, may be generated at the CdS surface. Gas chromatographic analysis of headspace gas during photosynthesis does detect trace amounts of H₂ roughly in the range of 1-10 ppm. While it is difficult to conclude the prevalence of this reaction pathway (as H₂ may be generated at CdS, but immediately consumed by M. thermoacetica before it escapes to the headspace), it demonstrates that this pathway is present to some extent.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified microorganism capable of photosynthesizing an organic compound from carbon dioxide, wherein the microorganism comprises a semiconductor nanoparticle on the surface of the microorganism.

2. A method for constructing the microorganism of claim 1, comprising: (a) introducing a nucleic acid encoding one or more biosynthetic enzymes into a microorganism wherein the one or more biosynthetic enzymes enable the microorganism to produce the organic compound, (b) introducing the microorganism into a solution comprising a semiconductor source, a carbon source, and a reducing agent, and (c) culturing the microorganism in the solution, such that the microorganism incorporates a semiconductor nanoparticle on the surface of the microorganism.

3. A method of producing an organic compound, comprising: (a) providing a genetically modified microorganism of claim 1, (b) exposing the microorganism to sufficient light to cause the semiconductor nanoparticle to generate hydrogen, such that the microorganism uses the generated hydrogen to produce the organic compound.
